(19) United States
(12) Patent Application Publication
(10) Pub. No.: US 2025/0235458 A1
(43) Pub. Date: Jul. 24, 2025

(54) Benzimidazole derivatives for use in the treatment or prevention of a histiocytosis or a craniopharyngioma

(71) Applicants: UNIVERSITE CLAUDE BERNARD LYON 1, VILLEURBANNE (FR); CENTRE NATIONAL DE LA RECHERCHE SCIENTIFIQUE, PARIS (FR); INSTITUT NATIONAL DE LA SANTÉ ET DE LA RECHERCHE MÉDICALE, PARIS (FR); HOSPICES CIVILS DE LYON, LYON (FR); CENTRE LEON BERARD, LYON (FR)

(72) Inventors: Luc OTTEN, CRASSIER (CH); Toufic RENNO, CIVRIEUX D'AZERGUES (FR); Isabelle COSTE-INVERNIZZI, CHAZAY D'AZERGUES (FR); Stéphane GIRAUD, L'ISLE D'ABEAU (FR)

(73) Assignees: UNIVERSITE CLAUDE BERNARD LYON 1, VILLEURBANNE (FR); CENTRE NATIONAL DE LA RECHERCHE SCIENTIFIQUE, PARIS (FR); INSTITUT NATIONAL DE LA SANTÉ ET DE LA RECHERCHE MÉDICALE, PARIS (FR); HOSPICES CIVILS DE LYON, LYON (FR); CENTRE LEON BERARD, LYON (FR)

(21) Appl. No.: 18/699,257

(22) PCT Filed: Oct. 7, 2022
(86) PCT No.: PCT/EP2022/077910

§ 371 Date: Apr. 5, 2024

(30) Foreign Application Priority Data
Oct. 8, 2021 (EP) 21306418.1

(51) Int. Cl. A61K 31/5377; A61K 31/4184; A61K 31/422; A61K 31/4439; A61K 31/454; A61K 31/506; A61K 45/06; A61P 35/00

(52) U.S. Cl. CPC A61K 31/5377; A61K 31/4184; A61K 31/422; A61K 31/4439; A61K 31/454; A61K 31/506; A61K 45/06; A61P 35/00

(57) ABSTRACT

The present invention relates to benzimidazole derivatives and pharmaceutical compositions comprising such benzimidazole derivatives, for use in the treatment or prevention of a histiocytosis or a craniopharyngioma.

### TECHNICAL FIELD

The present invention relates to benzimidazole derivatives and pharmaceutical compositions comprising such benzimidazole derivatives, for use in the treatment or prevention of a histiocytosis or a craniopharyngioma.

### BACKGROUND

Histiocytoses are rare disorders characterized by the accumulation of macrophage-, dendritic cell- or monocyte-derived cells in various tissues and organs of children and adults. Since the first classification in 1987, a number of new findings concerning the cellular origins, molecular pathology and clinical features of histiocytic disorders have been identified.

In particular, following the discovery of recurrent mutations in Langerhans Cell Histiocytosis (LCH) in 2010 (Badalien-Very et al. [01]), the classification of histiocytoses based on histology, phenotype, molecular alterations and clinical and imaging features has been revised and includes 5 disease groups as follows (Emile et al. [02]):
(1) the “L” group relating to Langerhans-related histiocytoses, notably including a Langerhans cell histiocytosis (LCH), a Erdheim-Chester disease (ECD), a mixed form of Erdheim-Chester Disease and Langerhans cell histiocytosis (mixed ECD and LCH), and an indeterminate cell histiocytosis (ICH);
(2) the “C” group relating to cutaneous and mucocutaneous histiocytosis, notably including a xanthogranuloma histiocytosis such as a Juvenile Xanthogranuloma (JXG);
(3) the “M” groups relating to malignant histiocytoses, notably including primary and secondary malignant histiocytoses;
   In contrast to histiocytosis from all other groups (L, C, R and H), somatic mutations are observed with frequent chromosomal anomalies and presence of multiple mutations (Emile et al. [02]). In addition to mutations in MAPK pathway genes, mutations are found in CDKN2A and TP53 genes and rarely in NFxB pathway genes (Massoth et al. [28]).
(4) the “R” group relating to a Rosai-Dorfman disease (RDD) and miscellaneous non-cutaneous, non-Langerhans cell histiocytoses; and
(5) the “H” group relating to hemophagocytic lymphohistiocytosis (HLH) divided into two subgroups: primary (with known Mendelian inheritance) and secondary (reactive).

Since few years, LCH is defined as an inflammatory myeloid neoplasia (da Costa et al. [24], Kobayashi and Tojo [03]; Allen et al. [04]; Rodriguez-Galindo and Allen [05]). LCH is however not a malignant neoplasia or a cancer as LCH does not show gross genomic alterations (da Costa et al. [24]), genome instability (Chakraborty et al. [22]) and thus tumor progression, which is a key enabling characteristic of cancer (Hanahan and Weinberg [06]).

Recently, LCH as a myeloid neoplastic disorder has been described as being the result of misguided myeloid differentiation, with mutually exclusive somatic activating mutations in MAPK pathway genes being identified in approximately 85% of the lesions (Gulati and Allen [07]). Mutations in these genes are also found in other non-malignant histiocytic diseases from the L Group (Erdheim-Chester Disease ECD), the C group (Juvenile Xanthogranuloma JXG) and the R group (Rosai Dorfman Disease) (Emile et al. [02]; Gulati and Allen [07]; Wu et al. [08]).

Activation of MAPK with gene mutations in LCH is detected in various precursor cells of pathogenic myeloid cells found in histiocytosis lesions (Rodriguez-Galindo and Allen [05]; Gulati and Allen [07]). The activation of MAPK pathway in precursor cells is described as leading to enhanced myeloid differentiation, impaired migration, apoptosis inhibition and inflammation but no increased proliferation is observed in pathogenic myeloid cells found in histiocytosis lesions (Allen et al. [04]), thus further distinguishing LCH from cancer (Hanahan and Weinberg [06]).

Gene mutations activating the MAPK pathway often occur at the BRAF level (Michaloglou et al. [26]; Pisapia et al. [27]). However, the role of BRAF mutations in different diseases is variable since i) these mutations are found in both cancers and benign neoplasia, ii) they are poorly associated with malignant status of some neoplasia, iii) they can have a positive or negative prognostic value depending on cancer types and/or concomitant mutations, iv) they can induce proliferation, cell senescence or epilepsy, and v) they poorly correlate with downstream activation of ERK in benign lesions.

The importance of pathogenesis of L Group diseases driven by somatic activating mutations in MAPK pathway genes is supported by MAPK inhibitory drug offering treatments of LCH and of ECD with a near universal response but unfortunately non-curative (Rodriguez-Galindo and Allen [05]; Gulati and Allen [07]; Cohen-Aubart et al. 2017 [39]).

In view of the important inflammation found in LCH, alternative pathogenic mechanisms have been proposed like infection and inflammation (Berres et al 2015 [29]). Virus-related pathogenesis has been searched but is still unproven: after disregarded multiple viruses (such as EBV and herpesviruses), MCPyV is the latest published virus associated to LCH (Murakami et al. 2014 [30]) but its significance in pathogenesis of LCH is unclear (Berres et al 2015 BJH [29]; Murakami et al. 2015 [31]). MCPyV is a common virus viewed as a major risk for Merkel cell carcinoma where alterations are found in RB1, TP53, NOTCH and PI3K-AKT-mTOR pathways (Stachyra et al 2021, IJMS [32]), but MAPK signaling is absent with no phosphorylated ERK and no activating mutation in BRAF (V600E) gene is found in Merkel cell carcinoma (Houben et al. JID 2006 [33]), contrary to what has been established in LCH. In addition, copies of MCPyV are found in LCH at levels that are 30 to 500 lower than in Merkel cell carcinoma (Murakami et al. 2014 [30], Stachyra et al 2021, IJMS [32]). Moreover, MCPyV DNA is not found in pulmonary form of LCH (Jouenne et al 2020 [34]). Furthermore, the role of inflammation remains uncertain with unconfirmed causal role of interleukin-17 (Berres et al 2015 BJH [29]) and reports of variable and incomplete effect of IL-1 pathway blocking in ECD only (Cohen-Aubart et al. 2016 [35]), which is in sharp contrast with the very high response rate of therapies targeting MAPK pathway ((Rodriguez-Galindo and Allen [05]; Gulati and Allen [07]). It follows that somatic activating mutations in MAPK pathway genes occurring in various diseases in L group is currently the only established mechanism and the importance of MCPyV or inflammation is highly questionable and remains to be proven. Furthermore, a putative crosstalk between MAPK pathway and infection or inflammation in L group is entirely speculative.

Craniopharyngiomas are rare epithelial tumours that arise in the suprasellar region of the brain (Brastianos et al. [11]; Roque and Odia [12]). The two main disease subtypes are the adamantinomatous craniopharyngioma (ACP) observed in paediatric and adult populations and the papillary craniopharyngioma (PCP) mostly seen in adult population (Roque and Odia [12]; Gan [13]). Craniopharyngiomas with mixed ACP and PCP histology were described in 5% to 11% of cases in older series but “mixed craniopharyngiomas” are not part of World Health Organisation classification (Gan [13]). Frequently, severe morbidity occurs from tumour extension and therapeutic intervention (Brastianos et al. [11]). Permanent brain damages (optic nerves, pituitary gland, hypothalamus, ventricular system and brainstem) lead to visual loss, endocrine defect, neurobehavioral changes and headaches (Gan [13]).

In 2014, ACP and PCP subtypes were further differentiated with the discovery of somatic mutations in beta-catenin gene (CTNNB1) activating the WNT signaling in majority of ACP cases, and recurring mutations in BRAF (V600E) gene activating the MEK/MAPK pathway in most PCP cases (Brastianos et al. [11]; Gan [13]). Mutations in CTNNB1 and in BRAF are mutually exclusive with the exception of rare described cases (Brastianos et al. [11]; Larkin et al. [14]). Frequency of mutations in other gene exons is low alike the benign histology of these tumors. Clonal activating mutations in CTNNB1 and BRAF in ACP and PCP, respectively were then proposed to be critical events in the pathogenesis of these tumours (Brastianos et al. [11]).

In recent years, ACP and PCP tumors are described as “benign tumors in a malignant location” (Roque and Odia [12]). This description of the craniopharyngiomas reflects the benign histology features (Aylwin et al. [14]; Rao et al. [16]) and low mutation rate (Brastianos et al. [11]), and locally aggressive evolution (Rostami et al. [17]; Brastianos et al. [18]; Himes et al. [19]). ACP and PCP are viewed as benign tumours originating from Rathke's embryologic remnant (Juratli et al. [20]; Schlaffer et al. [21]). ACP and PCP tumors share a number of features with histiocytoses:
(1) Both diseases groups are classified as benign tumours with possible aggressive/severe clinical evolution (Kobayashi and Tojo [03]; Allen et al. [04]; Rodriguez-Galindo and Allen [05]; Roque and Odia [12]; Rostami et al. [17]; Brastianos et al. [18]; Himes et al. [19]).
(2) Activating mutation of BRAF gene (V600E) is found as the most frequent somatic mutation observed in subgroups of craniopharyngiomas and of histiocytoses, PCP (Brastianos et al. [11]) and LCH (Gulati and Allen [05]), respectively.
(3) Activating genetic mutation of BRAF and of other genes are mutually exclusive in craniopharyngiomas (Brastianos et al. [11]) and usually found at a single level of the MAPK pathway in histiocytoses (Chakraborty et al. [22]).
(4) Somatic mutations of other genes is low in both craniopharyngiomas and histiocytoses (Brastianos et al. [11]; Chakraborty et al. [22]).
(5) Somatic BRAF (V600E) mutation in brain is viewed as driving event of PCP (Brastianos et al. [11]) while somatic mutations of BRAF (V600E) is observed in brain regions of patients with LCH (Rodriguez-Galindo and Allen [05]) and can lead to neurodegenerative lesions (Rodriguez-Galindo and Allen [05]; Gulati and Allen [07]; Mass et al. [23]).
(6) Embryonic origin of pathogenic cells is evoked in both craniopharyngiomas (Juratli et al. [20]; Schlaffer et al. [21]) and in histiocytoses (Rodriguez-Galindo and Allen [05]; Mass et al. [23])

Treatments of LCH make use of conventional therapies, such as surgery, corticosteroids, mustard agents, thalidomide, MAPK inhibitors like BRAF inhibitors (e.g. vemurafenib, dabrafenib, or cobimetinib) or a combined chemotherapy (e.g. vinblastine-prednisone or MACOP-B, MACOP-B consisting in the combined use of methotrexate, doxorubicin (Adriamycin®), cyclophosphamide, vincristine (Oncovin®), prednisone et bleomycin) (Heisig et al. [25]; Gulati and Allen [07]; Rodriguez-Galindo and Allen [05]; Donadieu et al. [09]).

Treatments of craniopharyngioma make use of neurosurgery with or without adjuvant or salvage radiotherapy (Gan [13]). Patients frequently suffer from hypothalamic damages and both ACP and PCP subtypes tend to progress and to recur with further hypothalamo-pituitary damages (Gan [13]). Patients with PCP subtype with BRAF mutation V600E are treated with targeted drug therapies, such as vemurafenib, or dabrafenib, or dabrafenib with trametinib.

Unfortunately, current drug therapies of histiocytosis and craniopharyngioma are often not curative in patients with severe disease so that there still exists a need for new drug therapies for these diseases, notably having an improved efficacy, longer lasting efficacy, higher safety and/or being curative.

### SUMMARY OF THE INVENTION

The inventors observed for the first time the presence of the protein-protein complex ERK/MyD88 in histiocytosis lesions (see the examples), leading to the possibility to treat such a disease with an inhibitor of the ERK/MyD88 interaction, as well as craniopharyngioma which, as explained above, is a disease very similar to histiocytosis.

Thus, according to a first aspect, the present invention relates to a compound of following formula (I):

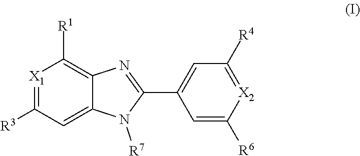

or a pharmaceutically acceptable salt thereof, a stereoisomer thereof or a mixture of stereoisomers thereof, wherein:
X₁ represents N or CR²;
X₂ represents N or CR⁵;
R¹ and R³ represent, independently of each other, H, (C1-C6)alkyl or halogen;
R² represents CN; a (C1-C6)alkyl group optionally substituted with one or more halogen atoms; an aryl or heteroaryl group optionally substituted with one or more groups selected from halo, (C1-C6)alkyl, OR21 and NR22R23; or CONR11R12; wherein
R11 represents H or (C1-C6)alkyl;
R12 represents a (C1-C6)alkyl, aryl, aryl-(C1-C6)alkyl or 5- or 6-membered heteroaryl group optionally substituted with one or more groups selected from halo, (C1-C6)alkyl, aryl, OR24 and NR25R26;
R21, R22, R23, R24, R25, and R26 represent, independently of one another, H or (C1-C6)alkyl;
R⁴ and R⁶ represent, independently of each other, H, halogen such as Cl, CN, NO₂, (C1-C6)alkyl, NR15COR16, NR17R18 or OR19, wherein:
R15 and R19 represent, independently of each other, H or (C1-C6)alkyl;
R16 represents (C1-C6)alkyl;
R17 and R18 represents H, (C1-C6)alkyl, aryl, or heteroaryl, such as H or (C1-C6)alkyl;
R⁵ represents NR13R14 wherein:
R13 represents H, R31 or COR32;
R14 represents H, R33 or COR34;
or R13 and R14 form together with the nitrogen atom bearing them a heterocycle optionally substituted with a (C1-C6)alkyl group;
R31, R32, R33 and R34 represent, independently of one another, a (C1-C6)alkyl, aryl, aryl-(C1-C6)alkyl or heteroaryl group optionally substituted with one or more groups selected from halo, (C1-C6)alkyl, OR27 and NR28R29;
R27, R28 and R29 represent, independently of one another, H or (C1-C6)alkyl; and
R⁷ represents H or (C1-C6)alkyl;
   for use in the treatment or prevention of a histiocytosis or a craniopharyngioma, preferably a histiocytosis.

The present invention relates also to the use of a compound of formula (I) as defined above, or a pharmaceutically acceptable salt thereof, a stereoisomer thereof or a mixture of stereoisomers thereof, for the treatment or prevention of a histiocytosis or a craniopharyngioma, preferably a histiocytosis.

The present invention relates also to the use of a compound of formula (I) as defined above, or a pharmaceutically acceptable salt thereof, a stereoisomer thereof or a mixture of stereoisomers thereof, for the manufacture of a medicinal product for use in the treatment or prevention of a histiocytosis or a craniopharyngioma, preferably a histiocytosis.

The present invention relates also to a method for treating or preventing a histiocytosis or a craniopharyngioma, preferably a histiocytosis, comprising administrating to a person in need thereof of an effective amount of a compound of formula (I) as defined above, or a pharmaceutically acceptable salt thereof, a stereoisomer thereof or a mixture of stereoisomers thereof.

According to a second aspect, the present invention relates to a pharmaceutical composition comprising at least one compound of formula (I) as defined above, or a pharmaceutically acceptable salt thereof, a stereoisomer thereof or a mixture of stereoisomers thereof, and a pharmaceutically acceptable carrier, for use in the treatment or prevention of a histiocytosis or a craniopharyngioma, preferably a histiocytosis.

The present invention relates also to the use of a pharmaceutical composition as defined above for the treatment or prevention of a histiocytosis or a craniopharyngioma, preferably a histiocytosis.

The present invention relates also to the use of a pharmaceutical composition as defined above for the manufacture of a medicinal product for use in the treatment or prevention of a histiocytosis or a craniopharyngioma, preferably a histiocytosis.

The present invention relates also to a method for treating or preventing a histiocytosis or a craniopharyngioma, preferably a histiocytosis, comprising administrating to a person in need thereof of an effective amount of a pharmaceutical composition as defined above.

According to a third aspect, the present invention relates to a product comprising (1) at least one compound of formula (I) as defined above and (2) at least one other active ingredient different from said compound of formula (I), as a combined preparation for a simultaneous, separate or sequential use or as a pharmaceutical composition,

for use in the treatment or prevention of a histiocytosis or a craniopharyngioma, preferably a histiocytosis,
said at least one other active ingredient being useful in the treatment or prevention of a histiocytosis or a craniopharyngioma.

### Definitions

The term “halogen” or “halo”, as used in the present invention, refers to a fluorine, chlorine, bromine or iodine atom.

The term “(C1-C6)alkyl”, as used in the present invention, refers to a straight or branched monovalent saturated hydrocarbon chain containing from 1 to 6 carbon atoms including, but not limited to, methyl, ethyl, n-propyl, iso-propyl, n-butyl, iso-butyl, sec-butyl, t-butyl, n-pentyl, n-hexyl, and the like.

The term “(C1-C6)haloalkyl”, as used in the present invention, refers to a (C1-C6)alkyl group as defined above substituted by one or more halogen atoms as defined above, such as chlorine and/or fluorine atoms. It can be in particular a trifluoromethyl group.

The term “(C1-C6)alkoxy-(C1-C6)alkyl”, as used in the present invention, refers to (C1-C6)alkoxy group as defined below bound to the molecule via a (C1-C6)alkyl group as defined above including, but not limited to CH₃—O—(CH₂)₂—.

The term “amino(C1-C6)alkyl”, as used in the present invention, refers to an amino group as defined below bound to the molecule via a (C1-C6)alkyl group as defined above.

The term “(C1-C6)alkoxy”, as used in the present invention, refers to a (C1-C6)alkyl group as defined above bound to the molecule via an oxygen atom, including, but not limited to, methoxy, ethoxy, n-propoxy, iso-propoxy, n-butoxy, iso-butoxy, sec-butoxy, t-butoxy, n-pentoxy, n-hexoxy, and the like.

The term “amino group”, as used in the present invention, refers to a group NH₂, NHAlk1 or NAlk1Alk2 in which Alk1 and Alk2, identical or different, represent a (C1-C6)-alkyl group as defined above. For example, it can be a dimethylamino group.

The term “aryl”, as used in the present invention, refers to an aromatic hydrocarbon group comprising preferably 6 to 10 carbon atoms and comprising one or more fused rings, such as, for example, a phenyl or naphthyl group. Advantageously, it will be a phenyl group.

The term “aryl-(C1-C6)alkyl”, as used in the present invention, refers to an aryl group as defined above bound to the molecule via a (C1-C6)alkyl group as defined above. In particular, an aryl-(C1-C6)alkyl group is a benzyl group.

The term “(C1-C6)alkyl-aryl”, as used in the present invention, refers to a (C1-C6)alkyl group as defined above bound to the molecule via an aryl group as defined above. In particular, a (C1-C6)alkyl-aryl group is a methyl-phenyl, ethyl-phenyl or propyl-phenyl (e.g. isopropryl-phenyl) group.

The term “haloaryl”, as used in the present invention, refers to an aryl group as defined above substituted by one or more halogen atoms as defined above, such as chlorine and/or fluorine atoms. In particular, it can be a chlorophenyl group (Cl-Ph-).

The term “(C1-C6)alkoxy-aryl”, as used in the present invention, refers to a (C1-C6)alkoxy group as defined above bound to the molecule via an aryl group as defined above. In particular, it can be an methoxyphenyl group (CH₃—O-Ph-).

The term “aminoaryl”, as used in the present invention, refers to an amino group as defined above bound to the molecule via an aryl group as defined above. In particular, it can be a dimethylaminophenyl group ((CH₃)₂N-Ph-).

The term “heteroaryl”, as used in the present invention, refers to an aromatic group comprising one or several, notably one or two, preferably one, fused hydrocarbon cycles in which one or several, notably one, two, three or four, advantageously one or two, carbon atoms each have been replaced with a heteroatom selected from a sulfur atom, an oxygen atom and a nitrogen atom, preferably selected from an oxygen atom and a nitrogen atom, in particular a nitrogen atom. It can be a benzothiazolyl, furyl, thienyl, pyrrolyl, oxazolyl, isoxazolyl, thiazolyl, isothiazolyl, imidazolyl, pyrazolyl, oxadiazolyl, thiadiazolyl, triazolyl, tetrazolyl, pyridyl, pyridazinyl, pyrimidinyl, pyrazinyl, triazinyl, quinolyl, isoquinolyl, quinoxalyl or indolyl group. Preferably, the heteroaryl is a 5- or 6-membered heteroaryl.

The term “5- or 6-membered heteroaryl”, as used in the present invention, refers to an heteroaryl as defined above comprising one cycle having 5- or 6-membered. It can be a furyl, thienyl, pyrrolyl, oxazolyl, isoxazolyl, thiazolyl, isothiazolyl, imidazolyl, pyrazolyl, oxadiazolyl, thiadiazolyl, triazolyl, tetrazolyl, pyridyl, pyridazinyl, pyrimidinyl, pyrazinyl, or triazinyl group. Preferably, the heteroaryl is a 5-membered heteroaryl such as furyl, thienyl, pyrrolyl, oxazolyl, isoxazolyl, thiazolyl, isothiazolyl, imidazolyl, pyrazolyl, oxadiazolyl, thiadiazolyl, triazolyl, or tetrazolyl; in particular furyl, pyrrolyl, oxazolyl, isoxazolyl, imidazolyl, pyrazolyl, oxadiazolyl (such as 1,3,5-oxadiazolyl), triazolyl, or tetrazolyl.

The term “(C1-C6)alkyl-heteroaryl”, as used in the present invention, refers to a (C1-C6)alkyl group as defined above bound to the molecule via an heteroaryl group as defined above. In particular, it can be an ethylpyridyl group (C₂H₅-pyridyl-).

The term “haloheteroaryl”, as used in the present invention, refers to a heteroaryl group as defined above substituted by one or more halogen atoms as defined above, such as chlorine and/or fluorine atoms.

The term “(C1-C6)alkoxy-heteroaryl”, as used in the present invention, refers to a (C1-C6)alkoxy group as defined above bound to the molecule via an heteroaryl group as defined above. In particular, it can be an methoxypyridyl group (CH₃—O-pyridyl-).

The term “aminoheteroaryl”, as used in the present invention, refers to an amino group as defined above bound to the molecule via an heteroaryl group as defined above.

The term “heterocycle” as used in the present invention refers to a saturated, unsaturated or aromatic, preferably saturated, monocycle or polycycle (comprising fused, bridged or spiro rings), preferably monocycle comprising preferably 5 to 10, notably 5 or 6, atoms in each ring(s), in which the atoms of the ring(s) consist of carbon atoms and one or more, advantageously 1, 2, 3 or 4, and more advantageously 1 or 2, heteroatoms, such as a nitrogen, oxygen or sulphur atom, the remainder being carbon atoms. A heterocycle can be notably for example thienyl, furanyl, pyrrolyl, pyrrolidinyl, morpholinyl, piperidinyl or piperazinyl, in particular pyrrolidinyl, morpholinyl, piperidinyl or piperazinyl. Preferably, the heterocycle is a saturated 5- or 6-membered heterocycle, such as pyrrolidinyl, morpholinyl, piperidinyl or piperazinyl.

The expression “pharmaceutically acceptable” as used in the present invention is intended to mean what is useful to the preparation of a pharmaceutical composition/medicinal product, and what is generally safe and non toxic, for a pharmaceutical use.

The expression “pharmaceutically acceptable salt” is intended to mean, in the framework of the present invention, a salt of a compound which is pharmaceutically acceptable, as defined above, and which possesses the pharmacological activity of the corresponding compound.

The pharmaceutically acceptable salts comprise:
(1) acid addition salts formed with inorganic acids such as hydrochloric, hydrobromic, sulfuric, nitric and phosphoric acid and the like; or formed with organic acids such as acetic, benzenesulfonic, fumaric, glucoheptonic, gluconic, glutamic, glycolic, hydroxynaphtoic, 2-hydroxyethanesulfonic, lactic, maleic, malic, mandelic, methanesulfonic, muconic, 2-naphtalenesulfonic, propionic, succinic, dibenzoyl-L-tartaric, tartaric, p-toluenesulfonic, trimethylacetic, and trifluoroacetic acid and the like, and
(2) salts formed when an acid proton present in the compound is either replaced by a metal ion, such as an alkali metal ion, an alkaline-earth metal ion, or an aluminium ion; or coordinated with an organic or inorganic base. Acceptable organic bases comprise diethanolamine, ethanolamine, N-methylglucamine, triethanolamine, tromethamine, and the like. Acceptable inorganic bases comprise aluminium hydroxide, calcium hydroxide, potassium hydroxide, sodium carbonate and sodium hydroxide.

The term “stereoisomers” as used in the present invention refers to configurational stereoisomers and includes geometric isomers and optical isomers.

The geometric isomers, also called E/Z isomers or cis-trans isomers, result from the different position of substituents on a double C═C bond which can have a Z or E configuration, also called cis or trans configuration.

The optical isomers result from the different position in space of substituents or lone pair of electrons on an atom (such as a carbon or sulphur atom) comprising four different substituents (including potentially a lone pair of electron). This atom thus represents a chiral or asymmetric center. Optical isomers which are not mirror images of one another are thus designated as “diastereoisomers,” and optical isomers which are non-superimposable mirror images are designated as “enantiomers”.

In particular, a stereoisomer is an optical isomer and more particularly an enantiomer.

An equimolar mixture of two enantiomers of a chiral compound is designated as racemate or racemic mixture.

The expression “compound of formula (I)” as used in the present invention includes a compound of formula (Ia), (Ib), (Ic), (Id) or (Ie).

### DETAILED DESCRIPTION

### Compound According to the Invention

A compound according to the present invention is a compound of following formula (I):

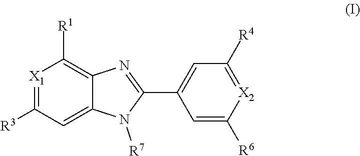

or a pharmaceutically acceptable salt thereof, a stereoisomer thereof or a mixture of stereoisomers thereof,
with X₁, X₂, R¹, R³, R⁴, R⁶ and R⁷ as defined above.

According to a particular embodiment, the compound according to the invention is a compound of following formula (Ia), (Ib), (Ic), (Id) or (Ie).

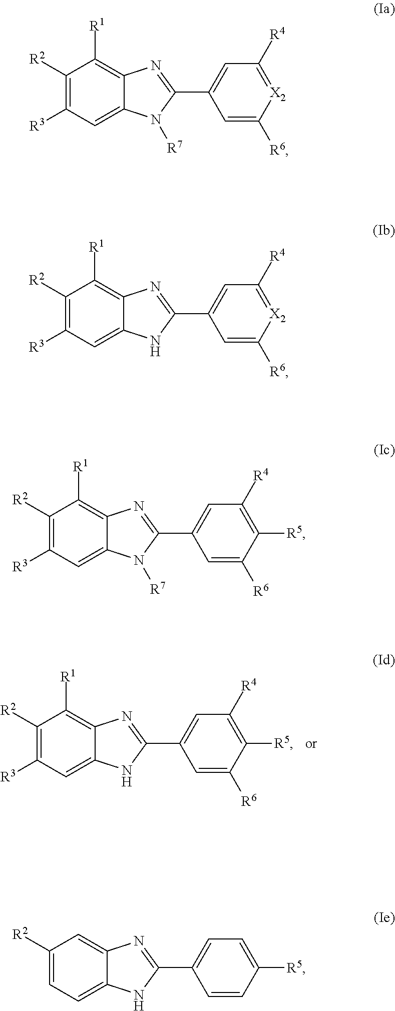

or a pharmaceutically acceptable salt thereof, a stereoisomer thereof or a mixture of stereoisomers thereof,

Advantageously, R¹ and R³ represent, independently of each other, H or (C1-C6)alkyl, such as H or CH₃, preferably H.

According to a particular embodiment, R⁴ and R⁶ represent, independently of each other, H, halogen such as Cl, CN, NO₂, NHCOR16, NHR18, or OR19. In particular, R⁴ represents H, halogen such as Cl, CN, NO₂, NHCOR16, NHR18, or OR19, and R⁶ represents H, OR19 or (C1-C6)alkyl.

Advantageously, R⁴ and R⁶ represent, independently of each other, H, OR19 or (C1-C6)alkyl, in particular H or (C1-C6)alkyl, such as H or CH₃, preferably H.

In particular, R¹, R³, R⁴, and R⁶ represent, independently of each other, H or (C1-C6)alkyl, such as H or CH₃, preferably H.

Advantageously, R⁷ represents H or CH₃, preferably H.

In particular, R¹, R³, R⁴, R⁶, and R⁷ represent, independently of each other, H or (C1-C6)alkyl, such as H or CH₃, preferably H.

According to a first embodiment, X₁ is N.

According to a second embodiment, X₁ is CR².

According to one particular embodiment, R² is CN, and preferably X₁ is CR².

According to another particular embodiment, R² is a (C1-C6)alkyl group optionally substituted with one or more halogen atoms such as F, and preferably X₁ is CR². R² can be more particularly CF₃.

According to another particular embodiment, R² is an aryl or heteroaryl group optionally substituted with one or more groups selected from halo, (C1-C6)alkyl, OR21 and NR22R23, and preferably X₁ is CR². Advantageously, it is a heteroaryl group optionally substituted with one or more groups selected from halo, (C1-C6)alkyl, OR21 and NR22R23. The heteroaryl group is preferably a 5- or 6-membered heteroaryl, such as a 6-membered heteroaryl, e.g. pyridyl, pyridazinyl, pyrimidinyl, pyrazinyl, or triazinyl and more particularly pyrimidinyl. R² can be in particular a pyrimidinyl group.

According to another particular and preferred embodiment, R² is CONR11 R12, and preferably X₁ is CR², with R11 and R12 as defined above and in particular with:
R11 representing H or (C1-C6)alkyl, such as H or CH₃, preferably H; and
R12 representing a (C1-C6)alkyl, aryl, or 5- or 6-membered heteroaryl group optionally substituted with one or more groups selected from halo, (C1-C6)alkyl, aryl, OR24 and NR25R26, in particular selected from halo, (C1-C6)alkyl, OR24 and NR25R26; such as (C1-C6)alkyl, aryl, heteroaryl, halo(C1-C6)alkyl, haloaryl, haloheteroaryl, (C1-C6)alkyl-aryl, (C1-C6)alkyl-heteroaryl, aryl-(C1-C6)alkyl, (C1-C6)alkoxy-(C1-C6)alkyl, (C1-C6)alkoxy-aryl, (C1-C6)alkoxy-heteroaryl, amino(C1-C6)alkyl, aminoaryl, or aminoheteroaryl; in particular (C1-C6)alkyl, aryl, heteroaryl, haloaryl, (C1-C6)alkyl-aryl, (C1-C6)alkyl-heteroaryl, aryl-(C1-C6)alkyl, (C1-C6)alkoxy-(C1-C6)alkyl, (C1-C6)alkoxy-aryl, (C1-C6)alkoxy-heteroaryl, or aminoaryl, the heteroaryl being a 5- or 6-membered heteroaryl.

Advantageously, R11 represents H or (C1-C6)alkyl, such as H or CH₃, preferably H, and R12 represents an aryl, or 5- or 6-membered heteroaryl group optionally substituted with one or more groups selected from halo, (C1-C6)alkyl, aryl, OR24 and NR25R26, in particular selected from halo, (C1-C6)alkyl, OR24 and NR25R26; such as aryl, heteroaryl, haloaryl, haloheteroaryl, (C1-C6)alkyl-aryl, (C1-C6)alkyl-heteroaryl, (C1-C6)alkoxy-aryl, (C1-C6)alkoxy-heteroaryl, aminoaryl, or aminoheteroaryl; in particular aryl, heteroaryl, halo-aryl, (C1-C6)alkyl-aryl, (C1-C6)alkyl-heteroaryl, aryl-(C1-C6)alkyl, (C1-C6)alkyl-heteroaryl, (C1-C6)alkoxy-aryl, (C1-C6)alkoxy-heteroaryl, or aminoaryl, the heteroaryl being a 5- or 6-membered heteroaryl. The aryl can be a phenyl and the 5- or 6-membered heteroaryl group can be furyl, thienyl, pyrrolyl, pyridyl, oxazolyl, isoxazolyl, thiazolyle, isothiazolyl, imidazolyl, pyrazolyl, oxadiazolyl, thiadiazolyl, triazolyl, tetrazolyl, pyridazinyl, pyrimidinyl, pyrazinyl, or triazinyl. Preferably, the 5- or 6-membered heteroaryl group is a 6-membered heteroaryl group such as pyridyl, pyridazinyl, pyrimidinyl, pyrazinyl, or triazinyl, preferably a pyridyl.

Advantageously, R11 represents H or (C1-C6)alkyl, such as H or CH₃, preferably H, and R12 represents a 5- or 6-membered heteroaryl group optionally substituted with one or more groups selected from halo, (C1-C6)alkyl, aryl, OR24 and NR25R26, in particular selected from halo, (C1-C6)alkyl, OR24 and NR25R26; such as heteroaryl, haloheteroaryl, (C1-C6)alkyl-heteroaryl, (C1-C6)alkoxy-heteroaryl, or aminoheteroaryl; in particular heteroaryl, (C1-C6)alkyl-heteroaryl, or (C1-C6)alkoxy-heteroaryl. The 5- or 6-membered heteroaryl group can be furyl, thienyl, pyrrolyl, pyridyl, oxazolyl, isoxazolyl, thiazolyle, isothiazolyl, imidazolyl, pyrazolyl, oxadiazolyl, thiadiazolyl, triazolyl, tetrazolyl, pyridazinyl, pyrimidinyl, pyrazinyl, or triazinyl. Preferably, the 5- or 6-membered heteroaryl group is a 6-membered heteroaryl group such as pyridyl, pyridazinyl, pyrimidinyl, pyrazinyl, or triazinyl, preferably a pyridyl.

Preferably, R11 represents H or (C1-C6)alkyl, such as H or CH₃, preferably H, and R12 represents a pyridyl, a (C1-C6)alkoxyl-pyridyl group (e.g. methoxypyridyl or ethoxypyridyl) or a (C1-C6alkyl)-pyridyl group (e.g. methylpyridyl or ethylpyridyl).

According to a third embodiment, X₂ is N.

According to a fourth embodiment, X₂ is CR⁵ with R⁵ representing NR13R14 with R13 and R14 as defined above and in particular with:
R13 representing H or (C1-C6)alkyl; and
R14 representing H, R33 or COR34, with R33 and R34 as defined above, and in particular with R33 representing a (C1-C6)alkyl group optionally substituted with one or more groups selected from halo, OR27 and NR28R29 and R34 representing a aryl or heteroaryl group optionally substituted with one or more groups selected from halo, (C1-C6)alkyl, OR27 and NR28R29,
or R13 and R14 forming together with the nitrogen atom bearing them a heterocycle optionally substituted with a (C1-C6)alkyl group.

According to one particular and preferred embodiment, R13 represents H or R31, in particular H or (C1-C6)alkyl, and R14 represents H or R33 or R13 and R14 form together with the nitrogen atom bearing them a heterocycle, preferably a saturated heterocycle, optionally substituted with a (C1-C6)alkyl group. In particular, R13 represents H or R31, in particular H or (C1-C6)alkyl, and R14 represents H or R33. Advantageously, R31 and R33 represent, independently of one another, a (C1-C6)alkyl group optionally substituted with one or more groups selected from halo, OR27 and NR28R29, in particular selected from OR27 and NR28R29; preferably a (C1-C6)alkyl group. Preferably, R31 represents a (C1-C6)alkyl group and R33 represents a (C1-C6)alkyl group optionally substituted with one or more groups selected from halo, OR27 and NR28R29, in particular selected from OR27 and NR28R29; preferably a (C1-C6)alkyl group. The heterocycle is preferably a saturated 5- or 6-membered heterocycle, such as pyrrolidinyl, morpholinyl, piperidinyl or piperazinyl.

According to another particular and preferred embodiment, R13 represents H or R31, in particular H or (C1-C6)alkyl, preferably H, and R14 represents COR34, with R34 as defined above. Advantageously, R34 represents an aryl or heteroaryl group optionally substituted with one or more groups selected from halo, (C1-C6)alkyl, OR27 and NR28R29. Preferably, the aryl is a phenyl and the heteroaryl is a 5- or 6-membered heteroaryl, more particularly a 5-membered heteroaryl, such as furyl, thienyl, pyrrolyl, oxazolyl, isoxazolyl, thiazolyl, isothiazolyl, imidazolyl, pyrazolyl, oxadiazolyl, thiadiazolyl, triazolyl, or tetrazolyl; in particular furyl, pyrrolyl, oxazolyl, isoxazolyl, imidazolyl, pyrazolyl, oxadiazolyl (such as 1,3,5-oxadiazolyl), triazolyl, or tetrazolyl.

Preferably, R13 and R14 each represent, independently of one another, H, or (C1-C6)alkyl, such as H or CH₃.

Advantageously, X₁ is CR² and/or X₂ is CR⁵ and preferably, X₁ is CR² and X₂ is CR⁵ with R² and R⁵ as defined above according to any one of the above-mentioned embodiments.

It is understood, that any one of the above-described embodiments can be combined with any other above-described embodiment. For example, it could be envisaged to combine a particular embodiment relative to R², with another particular embodiment relative to R⁵.

According to a first preferred embodiment, X₁ is CR² and X₂ is CR⁵, wherein:
R² is CONR11R12 with R11 representing H or (C1-C6)alkyl, such as H or CH₃, preferably H, and R12 representing a 5- or 6-membered heteroaryl group optionally substituted with one or more groups selected from halo, (C1-C6)alkyl, OR24 and NR25R26; and
R⁵ is NR13R14 with R13 representing H or R31 and R14 representing H or R33 or R13 and R14 forming together with the nitrogen atom bearing them a heterocycle, preferably a saturated heterocycle, optionally substituted with a (C1-C6)alkyl group, with R31 and R33 as defined above, according to one of the above-mentioned embodiments.

According to a second preferred embodiment, X₁ is CR² and X₂ is CR⁵, wherein:
R² is CONR11R12 with R11 representing H or (C1-C6)alkyl, such as H or CH₃, preferably H, and R12 representing a 5- or 6-membered heteroaryl group optionally substituted with one or more groups selected from halo, (C1-C6)alkyl, OR24 and NR25R26; and
R⁵ is NR13R14 with R13 representing H or R31 and R14 representing H or R33, with R31 and R33 as defined above, according to one of the above-mentioned embodiments.

According to a third preferred embodiment, X₁ is CR² and X₂ is CR⁵, wherein:
R² is CONR11R12 with R11 representing H or (C1-C6)alkyl, such as H or CH₃, preferably H, and R12 representing a 5- or 6-membered heteroaryl group optionally substituted with one or more groups selected from halo, (C1-C6)alkyl, OR24 and NR25R26; and
R⁵ is NR13R14 with R13 and R14 each representing, independently of one another, H, or (C1-C6)alkyl, such as H or CH₃.

According to a fourth preferred embodiment, X₁ is CR² and X₂ is CR⁵, wherein:
R² is CONR11 R12 with R11 representing H and R12 representing a pyridyl group optionally substituted with one or more groups selected from halo, (C1-C6)alkyl, OR24 and NR25R26; and
R⁵ is NR13R14 with R13 and R14 each representing, independently of one another, H, or (C1-C6)alkyl, such as H or CH₃.

In the above-mentioned four preferred embodiments, R¹, R³, R⁴, R⁶ and R⁷ each represent advantageously H.

In the above-mentioned four preferred embodiments, the 5- or 6-membered heteroaryl group can be furyl, thienyl, pyrrolyl, pyridyl, oxazolyl, isoxazolyl, thiazolyle, isothiazolyl, imidazolyl, pyrazolyl, oxadiazolyl, thiadiazolyl, triazolyl, tetrazolyl, pyridazinyl, pyrimidinyl, pyrazinyl, or triazinyl. Preferably, the 5- or 6-membered heteroaryl group is a 6-membered heteroaryl group such as pyridyl, pyridazinyl, pyrimidinyl, pyrazinyl, or triazinyl, preferably a pyridyl.

In the above-mentioned four preferred embodiments, the heterocycle is preferably a saturated 5- or 6-membered heterocycle, such as pyrrolidinyl, morpholinyl, piperidinyl or piperazinyl.

Advantageously the compound according to the invention is chosen among the following compounds:

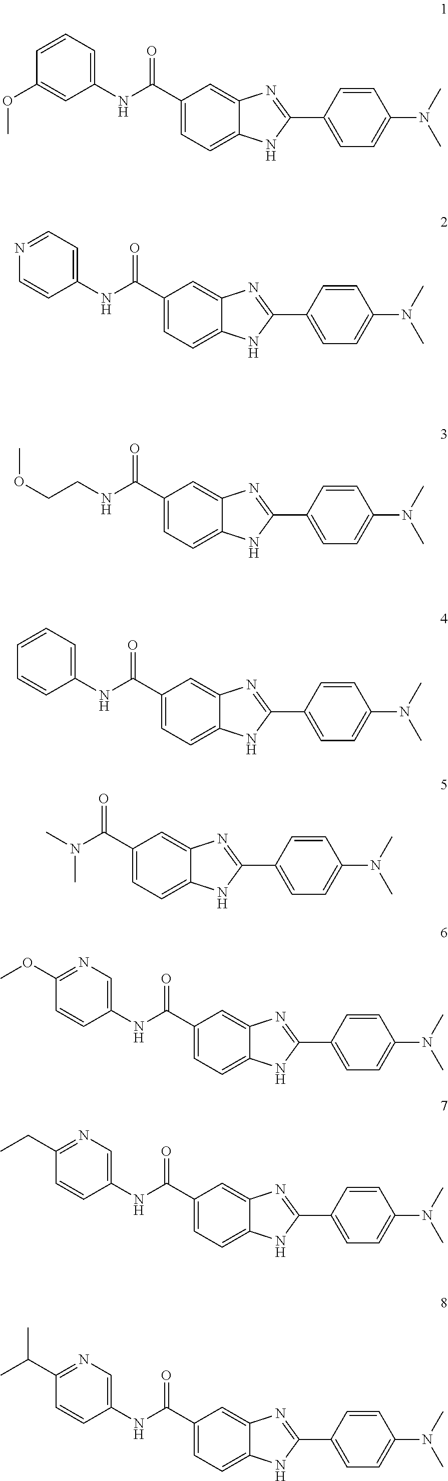

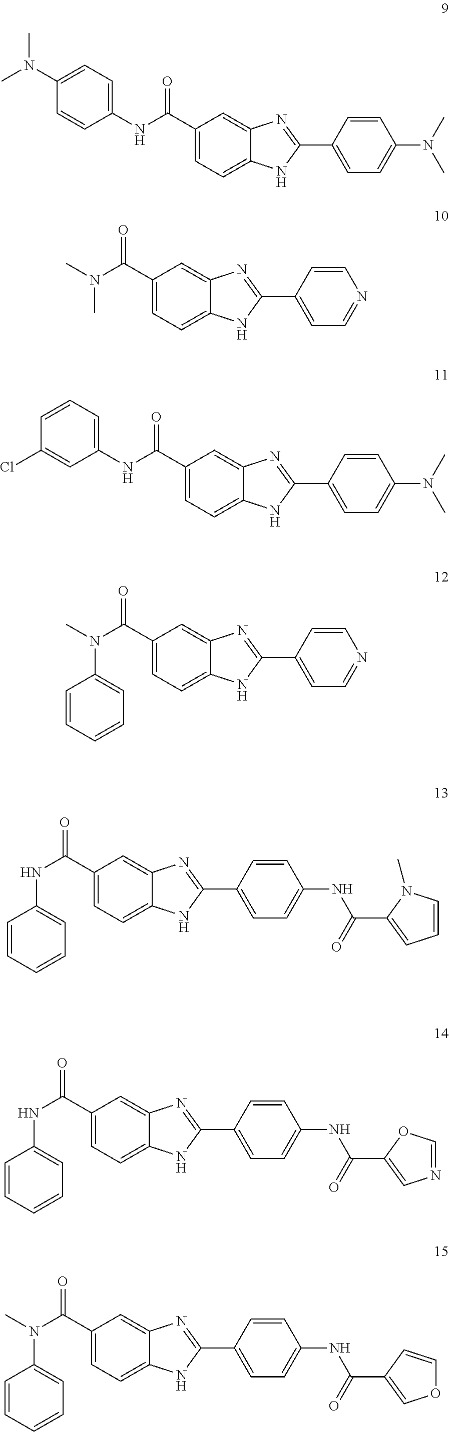

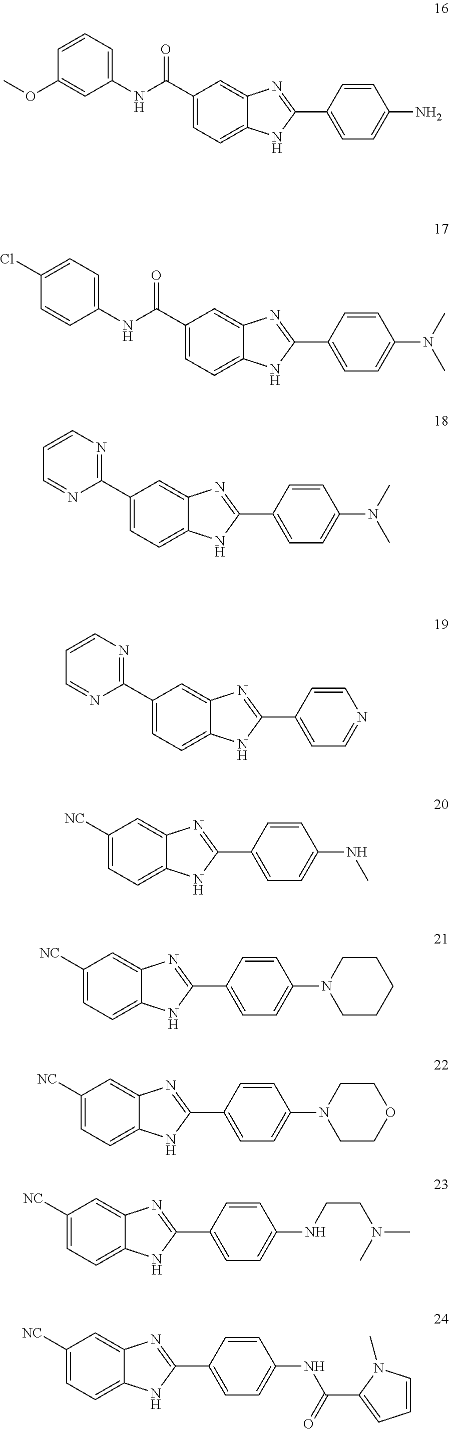

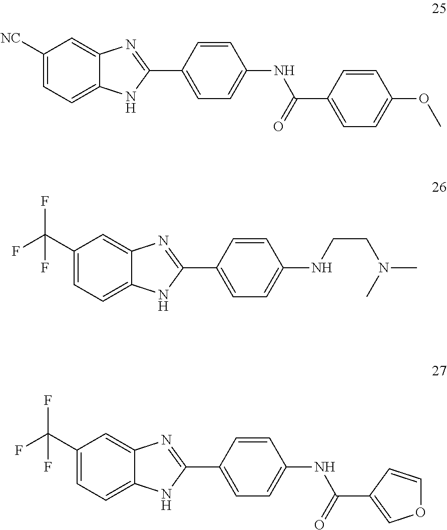 [SMILES: COc1ccc(C(=O)Nc2ccc(-c3nc4cc(C#N)ccc4[nH]3)cc2)cc1 ; CN(C)CCNc1ccc(-c2nc3cc(C(F)(F)F)ccc3[nH]2)cc1 ; O=C(Nc1ccc(-c2nc3cc(C(F)(F)F)ccc3[nH]2)cc1)c1ccoc1]

and the pharmaceutically acceptable salts thereof.

Preferably, the compound according to the invention is chosen among compounds 2, 6, and 7 and the pharmaceutically acceptable salts thereof.

The compounds according to the invention can be prepared as disclosed in WO2018/054989 ([010]).

### Pharmaceutical Composition According to the Invention

The pharmaceutical composition according to the invention comprises at least one compound according to the invention as defined above and a pharmaceutically acceptable carrier.

The pharmaceutical composition of the invention can be intended to enteral (e.g. oral, sublingual, buccal, rectal, vaginal, etc.), parenteral (e.g. subcutaneous, intramuscular, intravenous, intraocular, intraperitoneal, intracranial, intrathecal, etc.) or topical (e.g. transdermal) administration, preferably intravenous, oral, sublingual, subcutaneous, or topical administration. The active ingredient can be administered in unit forms for administration, mixed with conventional pharmaceutically acceptable carriers, to animals, preferably mammals including humans.

For oral administration, the pharmaceutical composition can be in a solid or liquid (solution or suspension) form.

A solid composition can be in the form of tablets, capsules, powders, granules and the like. In tablets, the active ingredient can be mixed with pharmaceutical vehicle(s) such as gelatin, starch, lactose, magnesium stearate, talc, gum arabic and the like before being compressed. The tablets may be further coated, notably with sucrose or with other suitable materials, or they may be treated in such a way that they have a sustained or delayed activity. In powders or granules, the active ingredient can be mixed or granulated with dispersing agents, wetting agents or suspending agents and with flavor correctors or sweeteners. In capsules, the active ingredient can be introduced into soft or hard capsules in the form of a powder or granules such as mentioned previously or in the form of a liquid composition such as mentioned below.

A liquid composition (including a gel) can contain the active ingredient together with a sweetener, a taste enhancer or a suitable coloring agent in a solvent such as water. The liquid composition can also be obtained by suspending or dissolving a powder or granules, as mentioned above, in a liquid such as water, juice, milk, etc. It can be for example a syrup or an elixir.

For sublingual (under the tongue) or buccal (between the gums and the cheek) administration, the pharmaceutical composition can be in a solid or liquid (solution or suspension) form.

A solid composition can be notably in the form of tablets, gelatin capsules, powders or granules as defined above for oral administration. It can be also in the form of a film.

A liquid composition can be as defined previously for oral administration. It can be administered in the form of a spray or drops.

For rectal or vaginal administration, suppositories or ovules can be prepared with binders which melt at rectal or vaginal temperature, for example cocoa butter or polyethylene glycols.

For parenteral administration, the composition can be in the form of an aqueous suspension or solution which may contain dispersing agents, wetting agents or suspending agents. The composition is advantageously sterile. It can be in the form of an isotonic solution (in particular in comparison to blood).

The compounds according to the invention can be used in a pharmaceutical composition at a dose ranging from 0.01 mg to 1000 mg a day, administered in only one dose once a day or in several doses along the day, for example twice a day in equal doses. The daily administered dose is advantageously comprised between 1 mg and 500 mg, and more advantageously between 10 mg and 200 mg. However, it can be necessary to use doses out of these ranges, which could be noticed by the person skilled in the art.

According to a particular embodiment, the pharmaceutical composition according to the invention further comprises at least one other active ingredient different from the compound according to the invention.

The at least one other active ingredient is preferably useful in the treatment or prevention of a histiocytosis or a craniopharyngioma, such as a corticosteroid (e.g. prednisone or dexamethasone), a mustard agent (e.g. cyclophosphamide or melphalan), a MAPK inhibitor like a BRAF inhinitor (e.g. vemurafenib, dabrafenib, or cobimetinib), thalidomide, or a combined chemotherapy such as vinblastine-prednisone or MACOP-B.

### Product According to the Invention

The product according to the invention comprises:
(1) at least one compound according to the invention as defined above, and
(2) at least one other active ingredient different from said compound of formula (I).

Said product is:
either a combined preparation for a simultaneous, separate or sequential use, i.e. the two active ingredients (1) and (2) are contained in two different pharmaceutical compositions that are administered to the person in need thereof simultaneously, separately or sequentially,
or a pharmaceutical composition, i.e. the two active ingredients (1) and (2) are contained in the same pharmaceutical composition.
   the treatment or prevention of a histiocytosis or a craniopharyngioma.

The at least one other active ingredient (2) is preferably useful in the treatment or prevention of a histiocytosis or a craniopharyngioma, such as a corticosteroid (e.g. prednisone or dexamethasone), a mustard agent (e.g. cyclophosphamide or melphalan), a MAPK inhibitor like a BRAF inhinitor (e.g. vemurafenib, dabrafenib, or cobimetinib), thalidomide, or a combined chemotherapy such as vinblastine-prednisone or MACOP-B.

### Pharmaceutical Application

The compound according to the invention, the pharmaceutical composition according to the invention and the product according to the invention are used in the treatment or prevention of a histiocytosis or a craniopharyngioma, preferably a histiocytosis.

According to a preferred embodiment, the histiocytosis is a non-malignant histiocytosis, preferably with benign histology features and without dysfunction in lymphocyte and/or natural killer cytotoxicity, features that are shared by the L, C and R groups of histiocytosis and by the secondary forms of H group. Preferably, the histiocytosis is selected from L, C and R groups, in particular from a Langerhans cell histiocytosis (LCH), a Erdheim-Chester disease (ECD), a mixed form of Erdheim-Chester Disease and Langerhans cell histiocytosis (mixed ECD and LCH), an indeterminate cell histiocytosis (ICH) (all from L group), a xanthogranuloma histiocytosis (XG) such as a Juvenile Xanthogranuloma (JXG) (from C group), and a Rosai-Dorfman disease (from R group). The histiocytosis may also be selected from a Langerhans cell histiocytosis (LCH), an indeterminate cell histiocytosis (ICH) (all from L group), a xanthogranuloma histiocytosis (XG) such as a Juvenile Xanthogranuloma (JXG) (from C group), and a Rosai-Dorfman disease (from R group). More preferably, the histiocytosis is from L group, preferably selected from a Langerhans cell histiocytosis (LCH), a Erdheim-Chester disease (ECD), a mixed form of Erdheim-Chester Disease and Langerhans cell histiocytosis (mixed ECD and LCH), and an indeterminate cell histiocytosis (ICH) or selected from a Langerhans cell histiocytosis (LCH) and an indeterminate cell histiocytosis (ICH); most preferably the histiocytosis is a Langerhans cell histiocytosis (LCH).

The histiocytosis (in general or any subgroup defined in the preceding paragraph) may or not contain one or more activating mutations in the RAS-MAPK pathway. In particular, the histiocytosis (in general or any subgroup defined in the preceding paragraph) may or not contain one or more activating mutations in genes selected from KRAS, NRAS, BRAF, ARAF, CRAF, MEK and ERK. Notably, the histiocytosis (in general or any subgroup defined in the preceding paragraph) may or not contain a BRAF V600E mutation.

By “activating mutation” in any gene of the RAS-MAPK pathway, it is referred to a mutation in the gene of interest that results in constitutive activation of the RAS-MAPK pathway, leading to constitutive ERK phosphorylation. Activating mutations in the RAS-MAPK pathway are known in the art. Table 1 below presents non-limiting examples of such activating mutations.

TABLE 1
Examples of known activating mutations in MAPK pathway.

| Gene | Known activating mutations |
|---|---|
| NRAS | Q61R (Diamond et al, 2013 [36]) |
| ARAF | S214A, A225V, P539H (Diamond et |
| | al, 2016 [37]) |
| BRAF | V600E, 600DLAT (insertion of amino |
| | acids DLAT at position 600; see Satoh |
| | et al. 2012 [38]), |
| MEK1 (MAP2K1) | K57E, del58-62, S123T, E144K |
| | (Diamond et al, 2016 [37]) |

In an embodiment, the compound according to the invention, the pharmaceutical composition according to the invention and the product according to the invention are used in the treatment or prevention of any histiocytosis (preferably non-malignant), excepted for a BRAF V600E mutated Erdheim-Chester disease (ECD) or mixed form of Erdheim-Chester Disease and Langerhans cell histiocytosis (mixed ECD and LCH).

In another embodiment, the compound according to the invention, the pharmaceutical composition according to the invention and the product according to the invention are used in the treatment or prevention of any histiocytosis (preferably non-malignant), excepted for a BRAF V600E mutated histiocytosis.

In another embodiment, the compound according to the invention, the pharmaceutical composition according to the invention and the product according to the invention are used in the treatment or prevention of any histiocytosis (preferably non-malignant) that does not contain one of the activating mutations of Table 1 above.

According to a preferred embodiment, the craniopharyngioma is a papillary craniopharyngioma (PCP) or an adamantinomatous craniopharyngioma (ACP).

### BRIEF DESCRIPTION OF THE DRAWINGS

FIG. 1 represents a tissue section from a patient with Langerhans cells histiocytosis which has been stained with MyD88 and ERK1/2 antibodies. Some dots representing ERK1/2-MyD88 interactions are identified with white arrows.

FIG. 2 shows the effect of compound 6 according to the invention or vemurafenib (BRAF inhibitor) at 8 μM and by comparison with the vehicle (DMSO) on the confluence (in percent) during time (in hours) of MutuDC1940 cells.

FIG. 3 shows the effect of compound 6 according to the invention or vemurafenib (BRAF inhibitor) at 8 μM and by comparison with the vehicle (DMSO) on the number of dead cells (on 13 mm²) during time (in hours) of MutuDC1940 cells.

FIG. 4 shows the effect of compound 6 according to the invention at 8 μM in comparison to the vehicle (DMSO) on the production of CXCL2 (in pg/mL) after a 16 h or 24 h treatment of MutuDC1940 cells.

FIG. 5 shows the effect of compounds 2, 6, 7 and 17 according to the invention at 8 μM and by comparison with the vehicle (DMSO) on the confluence (in percent) during time (in hours) of MutuDC1940 cells.

FIG. 6 shows the effect of compounds 2, 6, 7 and 17 according to the invention at 8 μM and by comparison with the vehicle (DMSO) on the number of dead cells (on 13 mm²) during time (in hours) of MutuDC1940 cells.

FIG. 7 shows the effect of vemurafenib (BRAF inhibitor) at 0.8 μM and by comparison with the vehicle (DMSO) on the confluence (in percent) during time (in hours) of MutuDC1940 cells.

FIG. 8 shows the effect of vemurafenib (BRAF inhibitor) at 0.8 μM and by comparison with the vehicle (DMSO) on the number of dead cells (on 13 mm²) during time (in hours) of MutuDC1940 cells.

FIG. 9 shows the effect of trametinib (MEK inhibitor) at 10 nM and by comparison with the vehicle (DMSO) on the confluence (in percent) during time (in hours) of MutuDC1940 cells.

FIG. 10 shows the effect of trametinib (MEK inhibitor) at 10 nM and by comparison with the vehicle (DMSO) on the number of dead cells (on 13 mm²) during time (in hours) of MutuDC1940 cells.

FIG. 11 shows the effect of ulixertinib (ERK inhibitor) at 1 μM and by comparison with the vehicle (DMSO) on the confluence (in percent) during time (in hours) of MutuDC1940 cells.

FIG. 12 shows the effect of ulixertinib (ERK inhibitor) at 1 μM and by comparison with the vehicle (DMSO) on the number of dead cells (on 13 mm²) during time (in hours) of MutuDC1940 cells.

### EXAMPLES

### Example 1

### 1. Materials and Methods

### Proximity Ligation Assay:

Tissue sections from a patient with Langerhans cells histiocytosis were deparaffinized and peroxidase activity was blocked by incubating slides in 3% H₂O₂ solution. After antigen retrieval in boiling citrate buffer pH6, MyD88 and ERK1/2 protein interaction was detected using Proximity Ligation Assay Kit (Sigma) according to manufacturer's instructions. MyD88 antibody (Invitrogen) and ERK1/2 (Cell Signaling) were used at 1/750 dilution in antibody diluent buffer. Brown dots represent ERK1/2-MyD88 interactions.

### Proliferation and Cell Death Assay:

1.5×10⁴ cells (MutuDC1940) were plated onto 96 wells plates. Next day, cells were treated at 8 μM of compound 6 or vemurafenib or with the vehicle (DMSO) in presence of 0.3 μg/ml of propidium iodide (Sigma) to detect cell death. Proliferation and cell death were quantified by Incucyte® (Essen Biosciences) according to manufacturer's instructions over 48 h.

### CXCL2 Quantification Assay:

0.5×10⁶ cells (MutuDC1940) were plated onto 6-well plates. Next day, cells were treated with 8 μM of compound 6 or DMSO for 16 or 24 hours. Each condition was performed in triplicate. Supernatants were collected and murine CXCL2 was measured by ELISA according to the manufacturer's instructions (Invitrogen).

### 2. Results

The results obtained for the Proximity Ligation Assay are presented on FIG. 1. The dots (some of which are identified with white arrows on FIG. 1) represent ERK1/2-MyD88 interaction in the tissue section from a patient with Langerhans cells histiocytosis and demonstrate that such an ERK1/2-MyD88 interaction is present in human histiocytosis.

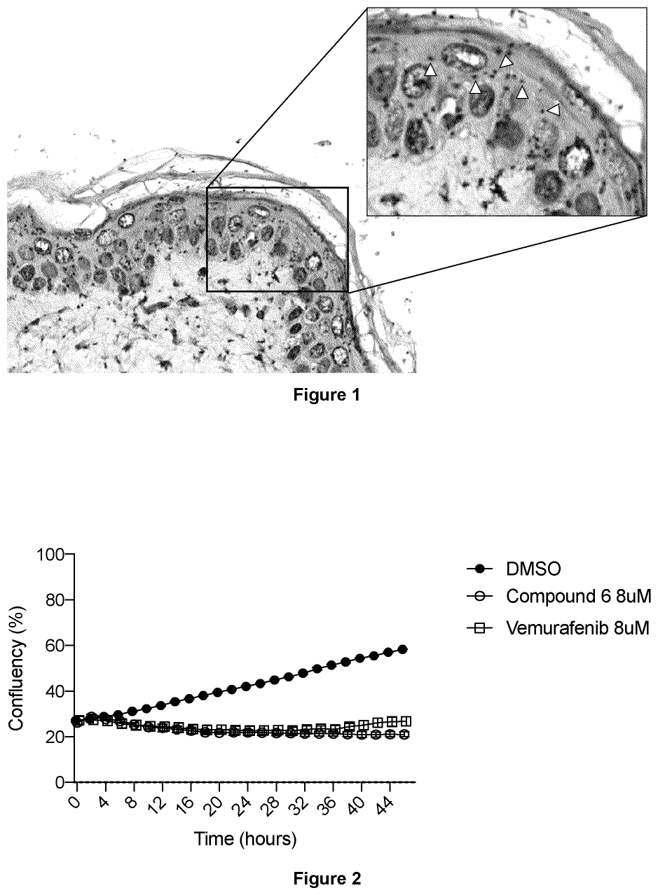

The results obtained for the proliferation assay are presented on FIG. 2. FIG. 2 presents the confluence, i.e. the percentage of the surface of the culture dish that is covered by the cells, observed during time for a culture of MutuDC1940 cells, a murine model of histiocytosis, treated at 8 μM with compound 6 according to the invention or with vemurafenib (a MAPK/BRAF inhibitor), or with the vehicle (DMSO). FIG. 2 shows that both compound 6 according to the invention and vemurafenib inhibits the proliferation of the cells.

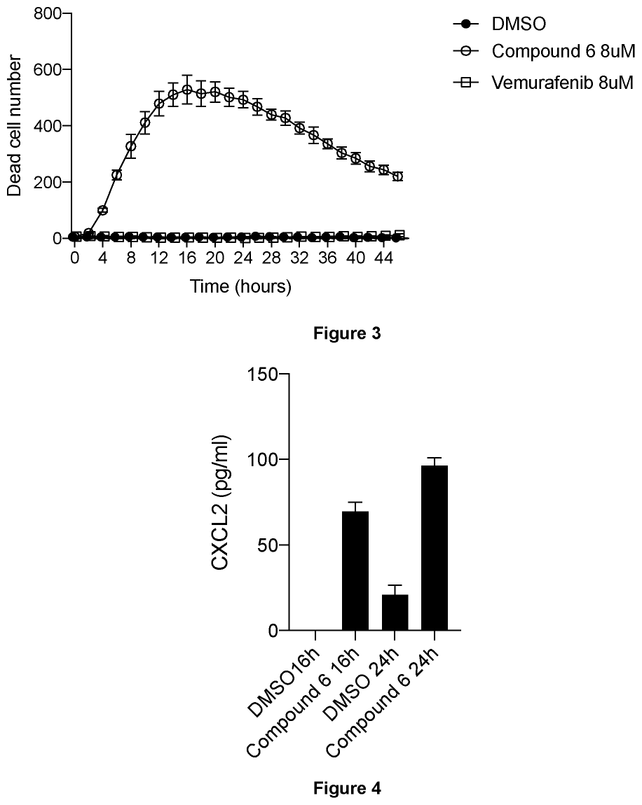

The results obtained for the cell death assay are presented on FIG. 3. FIG. 3 presents the number of dead cells observed during time for a culture of MutuDC1940 cells, a murine model of histiocytosis, treated at 8 μM with compound 6 according to the invention or with vemurafenib (a MAPK/BRAF inhibitor), or with the vehicle (DMSO). FIG. 3 shows that compound 6 induces the death of the cells, which is not the case of vemurafenib. This suggests that, contrary to known BRAF (a MAPK) inhibitor vemurafenib (which is known to be a non-curative treatment), compound 6 according to the invention and derivatives thereof as described herein might result in a curative treatment of histiocytosis, or at least a longer lasting treatment of histiocytosis.

The results of the CXCL2 quantification assay are presented on FIG. 4 and show that compound 6 according to the invention induces cytokine production in MutuDC1940 cells, a murine model of histiocytosis. This result confirms the relevance of this mouse cell line for human histiocytosis.

### Example 2: Effect of Further Compounds According to the Invention on Proliferation and Cell Death

The same experiments regarding cell proliferation (assessed by confluence in %) and cell death (assessed by number of dead cells on 13 mm²) as in Example 1 were repeated with additional ERK/MyD88 inhibitors of formula (I): compounds 2, 7 and 17.

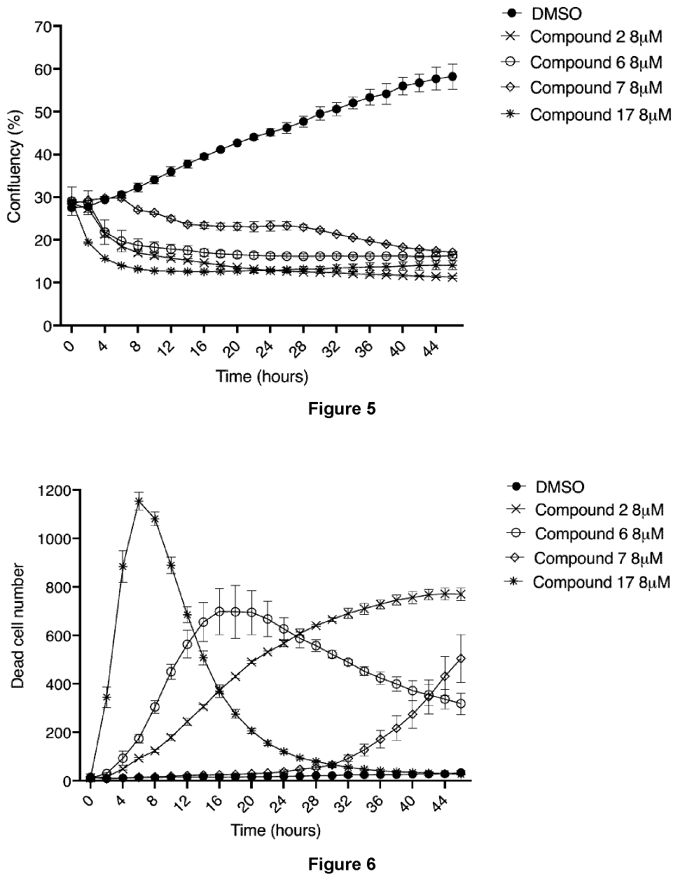

Results are presented in FIG. 5 (confluence in %) and FIG. 6 (number of dead cells on 13 mm²) and confirm that other compounds of formula (I) also not only inhibit proliferation but also induce the death of the histiocytosis cells.

This suggests that these compounds might result in a curative treatment of histiocytosis, or at least a longer lasting treatment of histiocytosis.

### Example 3: Effect of Other MAPK Inhibitors on Proliferation and Cell Death

The same experiments regarding cell proliferation (assessed by confluence in %) and cell death (assessed by number of dead cells on 13 mm²) as in Example 1 were repeated with a lower concentration, which corresponds to the IC50 of vemurafenib (BRAF inhibitor).

Trametinib (MEK inhibitor) and ulixertinib (ERK inhibitor), other inhibitors of the MAPK pathway, were used at the IC50 concentration of 10 nM and 1 μM respectively.

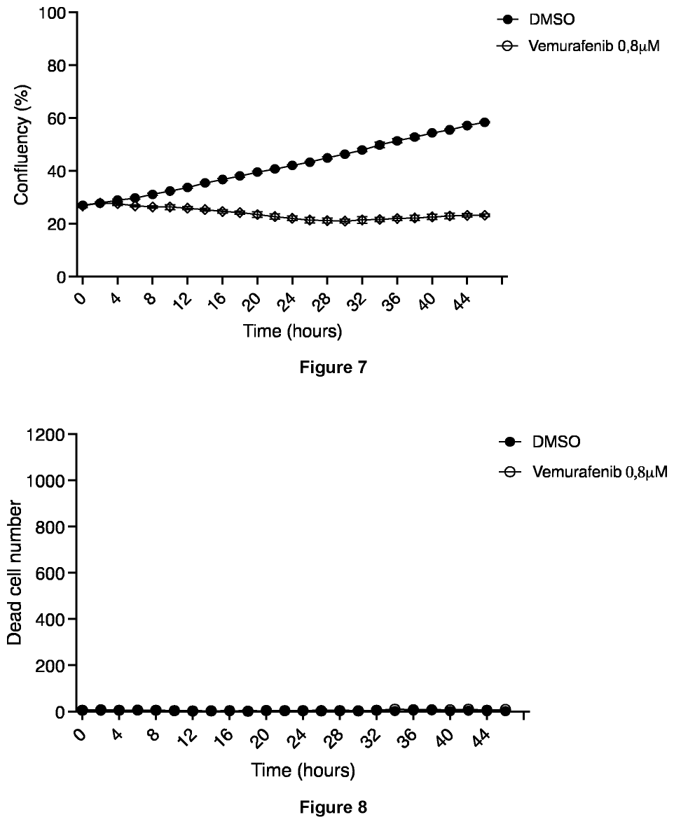

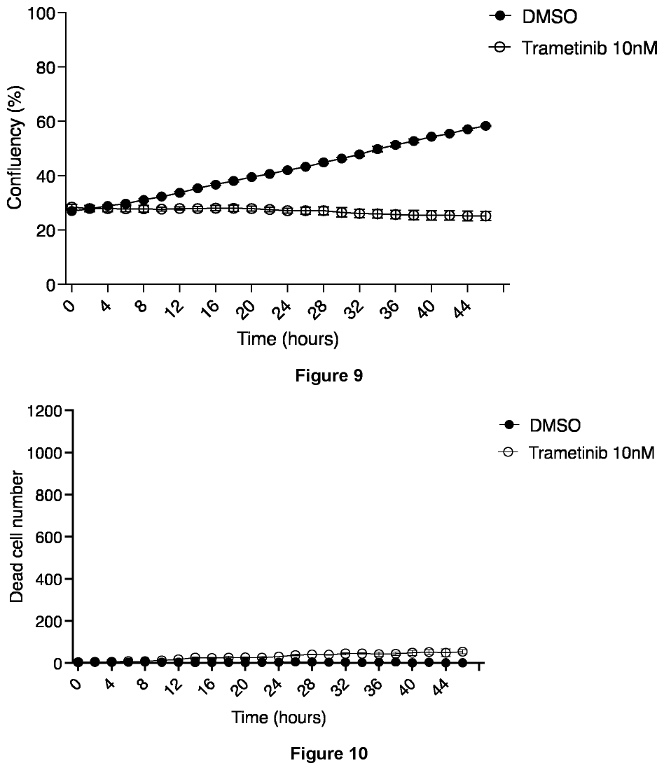

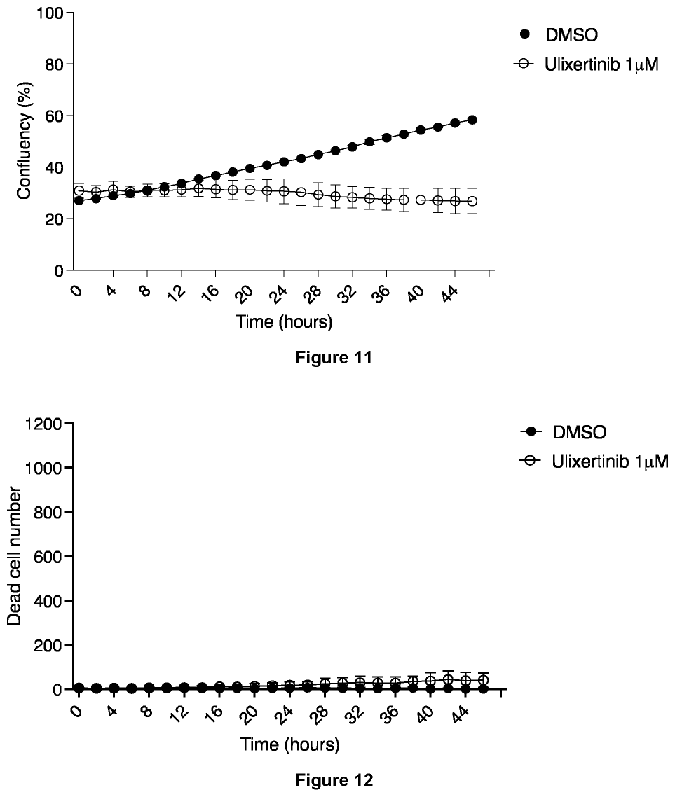

Results are presented in FIGS. 7 to 12 and show that, similarly to BRAF inhibitor verumafenib (see FIGS. 7-8), MEK inhibitor trametinib (see FIGS. 9-10) or ERK inhibitor ulixertinib (see FIGS. 11-12) only inhibit proliferation, with no effect on cell death, contrary to the compounds according to the invention.

### REFERENCES

[01] Badalien-Very et al. “Recurrent BRAF mutations in Langerhans cell histiocytosis” Blood 2010, 116(11), 1919-1923;
[02] Emilie et al. “Revised classification of histiocytoses and neoplasms of the macrophage-dendritic cell lineages” Blood 2016, 127(22), 2672-2681;
[03] Kobayashi and Tojo “Langerhans cell histiocytosis in adults: Advances in pathophysiology and treatment” Cancer Science 2018, 109, 3707-3713;
[04] Allen et al. “Langerhans-Cell Histiocytosis” N. Engl. J. Med. 2018, 379(9), 856-868;
[05] Rodriguez-Galindo and Allen “Langerhans cell histiocytosis” Blood 2020, 135(16), 1319-1331;
[06] Hanahan and Weinberg “Hallmarks of Cancer: The Next Generation” Cell 2011, 144, 646-674;
[07] Gulati and Allen “Langerhans cell histiocytosis: Version 2021” Hematological Oncology 2021, 39(S1), 15-23;
[08] Wu et al. “NRAS Mutations May Be Involved in the Pathogenesis of Cutaneous Rosai Dorfman Disease: A Pilot Study” Biology 2021, 10, 396, 1-15;
[09] Donadieu et al. “Vemurafenib for Refractory Multisystem Langerhans Cell Histiocytosis in Children: An International Observational Study” J. Clin. Oncol. 2019, 37(31), 2857-2866;
[10] WO2018/054989;
[11] Brastianos et al. “Exome sequencing identifies BRAF mutations in papillary craniopharyngiomas” Nat Genet. 2014, 46(2), 161-165;
[12] Roque and Odia “BRAF-V600E mutant papillary craniopharyngioma dramatically responds to combination BRAF and MEK inhibitors” CNS Oncol. 2017, 6(2), 95-99;
[13] Gan “Management of Craniopharyngiomas in the Era of Molecular Oncological Therapies: Not a Panacea” Journal of the Endocrine Society 2021, 5(7), 1-2;
[14] Larkin et al. “BRAF VE mutations are characteristic for papillary craniopharyngioma and may coexist with CTNNB1-mutated adamantinomatous craniopharyngioma” Acta Neuropathol 2014, 127, 927-929;
[15] Aylwin et al. “Pronounced response of papillary craniopharyngioma to treatment with vemurafenib, a BRAF inhibitor” Pituitary 2016, 19, 544-546.
[16] Rao et al. “Newly diagnosed papillary craniopharyngioma with BRAF V600E mutation treated with single-agent selective BRAF inhibitor dabrafenib: a case report” Oncotarget 2019, 10(57), 6038-6042;
[17] Rostami et al. “Recurrent papillary craniopharyngioma with BRAFV600E mutation treated with neoadjuvant-targeted therapy” Acta Neurochir 2017, 2217-2221;
[18] Brastianos et al. “Dramatic Response of BRAF V600E Mutant Papillary Craniopharyngioma to Targeted Therapy” JNCI J. Natl Cancer Inst 2016, 108(2), 1-5;
[19] Himes et al. “Recurrent papillary craniopharyngioma with BRAF V600E mutation treated with dabrafenib: case report” J Neurosurg 2018, 1-5;
[20] Juratli et al. “Targeted treatment of papillory craniopharyngiomas harboring BRAF V600E mutations” Cancer 2019, 125(17), 2910-2914;
[21] Schlaffer et al. “Rathke's Cleft Cyst as Origin of a Pediatric Papillary Craniopharyngioma” Frontiers in Genetics 2018, 9(49), 1-6;
[22] Chakraborty et al. “Mutually exclusive recurrent somatic mutations in MAP2K1 and BRAF support a central role for ERK activation in LCH pathogenesis” Blood 2014, 124(19), 3007-3015;
[23] Mass et al. “A somatic mutation in erythro-myeloid progenitors causes neurodegenerative disease” Nature 2017, 549(7672), 389-393;
[24] da Costa et al. “No genomic aberrations in Langerhans cell histiocytosis as assessed by diverse molecular technologies” Genes Chromosomes Cancer 2009, 48(3), 239-249;
[25] Heisig et al. “Vemurafenib in Langerhans cell histiocytosis: report of a pediatric patient and review of the literature” Oncotarget 2018, 9(31), 22236-22240;
[26] Michaloglou et al. “BRAFE600 in benign and malignant human tumours” Oncogene 2008, 27, 877-895;
[27] Pisapia et al. “BRAF: A Two-Faced Janus” Cells 2020, 9(12), 2549.
[28] Massoth et al., “Histiocytic and Dendritic Cell Sarcomas of Hematopoietic Origin Share Targetable Genomic Alterations Distinct from Follicular Dendritic Cell Sarcoma”, The Oncologist 2021, 26(7), e1263-e1272.
[29] Berres et al., “Progress in understanding the pathogenesis of Langerhans cellhistiocytosis: back to Histiocytosis X?”. British Journal of Haematology, 2015, 169(1), 3-13.
[30] Murakami et al. “Merkel cell polyomavirus DNA sequences in peripheral blood and tissues from patients with Langerhans cell histiocytosis”: Hum. Pathol. 2014 January; 45(1):119-26.
[31] Murakami et al. “Interleukin-1 loop model for pathogenesis of Langerhans cell histiocytosis” Cell Communication and Signaling 2015, 1-15
[32] Stachyra et al. “Merkel Cell Carcinoma from Molecular Pathology to Novel Therapies”. Int. J. Mol. Sci. 2021, 22(12), 6305.
[33] Houben et al. “Absence of Classical MAP Kinase Pathway Signalling in Merkel Cell Carcinoma”. J. Invest. Dermatol. 2006, 126(5), 1135-1142.
[34] Jouenne et al. “Lack of evidence for the involvement of Merkel cell polyomavirus in pulmonary Langerhans cell histiocytosis”. ERJ Open R. 2020, 6(2), 00230-2019.
[35] Cohen-Aubart et al. “Variability in the efficacy of the IL1 receptor antagonist anakinra for treating Erdheim-Chester disease”. Blood 2016, Volume 127(11), Pages 1509-1512.
[36] Diamond et al., “Detection of an NRAS mutation in Erdheim-Chester disease”, Blood 2013, 122(6), 1089-1091.
[37] Diamond et al., “Diverse and Targetable Kinase Alterations Drive Histiocytic Neoplasms”. Cancer Discov 2016, 6(2), 154-165.
[38] Satoh et al. “B-RAF mutant alleles associated with Langerhans cell histiocytosis, a granulomatous pediatric disease” [published correction appears in PLoS One. 2012; 7(6)]. PLoS One. 2012; 7(4), e33891.
[39] Cohen-Aubart et al. “Targeted therapies in 54 patients with Erdheim-Chester disease, including follow-up after interruption (the LOVE study). Blood. 2017; 130(11). 1377-1380.

1. A method of treating or preventing a histiocytosis or a craniopharyngioma, the method comprising administering to a subject a compound of following formula (I) 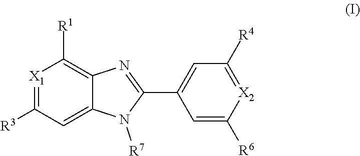
or a pharmaceutically acceptable salt thereof, a stereoisomer thereof or a mixture of stereoisomers thereof, wherein:
X₁ represents N or CR²;
X₂ represents N or CR⁵;
R¹ and R³ represent, independently of each other, H, (C1-C6)alkyl or halogen;
R² represents CN; a (C1-C6)alkyl group optionally substituted with one or more halogen atoms; an aryl or heteroaryl group optionally substituted with one or more groups selected from halo, (C1-C6)alkyl, OR21 and NR22R23; or CONR11R12; wherein
R11 represents H or (C1-C6)alkyl;
R12 represents a (C1-C6)alkyl, aryl, aryl-(C1-C6)alkyl or 5- or 6-membered heteroaryl group optionally substituted with one or more groups selected from halo, (C1-C6)alkyl, aryl, OR24 and NR25R26;
R21, R22, R23, R24, R25, and R26 represent, independently of one another, H or (C1-C6)alkyl;
R⁴ and R⁶ represent, independently of each other, H, halogen, CN, NO2, (C1-C6)alkyl, NR15COR16, NR17R18 or OR19, wherein:
R15 and R19 represent, independently of each other, H or (C1-C6)alkyl;
R16 represents (C1-C6)alkyl;
R17 and R18 represents H, (C1-C6)alkyl, aryl, or heteroaryl;
R⁵ represents NR13R14 wherein:
R13 represents H, R31 or COR32;
R14 represents H, R33 or COR34;
or R13 and R14 form together with the nitrogen atom bearing them a heterocycle optionally substituted with a (C1-C6)alkyl group;
R31, R32, R33 and R34 represent, independently of one another, a (C1-C6)alkyl, aryl, aryl-(C1-C6)alkyl or heteroaryl group optionally substituted with one or more groups selected from halo, (C1-C6)alkyl, OR27 and NR28R29;
R27, R28 and R29 represent, independently of one another, H or (C1-C6)alkyl; and
R⁷ represents H or (C1-C6)alkyl.

2. The method according to claim 1, wherein the compound is of following formula (Ia), (Ib), (Ic), (Id) or (Ie) 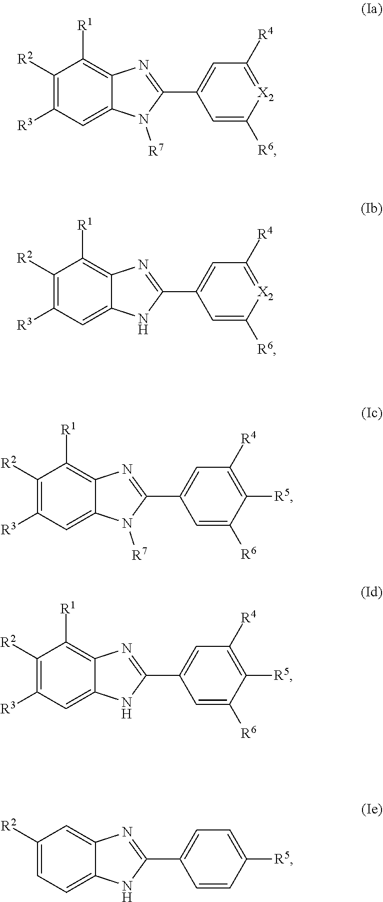
or a pharmaceutically acceptable salt thereof, a stereoisomer thereof or a mixture of stereoisomers thereof.

3. The method according to claim 1, wherein R¹, R³, R⁴, R⁶ and R⁷ each represent H.

4. The method according to claim 1, wherein X₁ represents CR².

5. The method according to claim 1, wherein X₂ represents CR⁵.

6. The method according to claim 1, wherein R² represents CONR11R12.

7. The method according to claim 1, wherein R11 represents H or CH₃, and R12 represents an aryl, or 5- or 6-membered heteroaryl group optionally substituted with one or more groups selected from halo, (C1-C6)alkyl, OR24 and NR25R26, wherein the aryl is a phenyl and the 5- or 6-membered heteroaryl group is a furyl, thienyl, pyrrolyl, pyridyl, oxazolyl, isoxazolyl, thiazolyle, isothiazolyl, imidazolyl, pyrazolyl, oxadiazolyl, thiadiazolyl, triazolyl, tetrazolyl, pyridazinyl, pyrimidinyl, pyrazinyl, or triazinyl.

8. The method according to claim 1, wherein R² represents CN; or
wherein R² represents a (C1-C6)alkyl group optionally substituted with one or several halogen atoms; or
wherein R² represents an aryl or heteroaryl group, optionally substituted with one or more groups selected from halo, (C1-C6)alkyl, OR21 and NR22R23.

9. The method according to claim 1, wherein R13 represents H or R31, and R14 represents H or R33, or wherein R13 and R14 form together with the nitrogen atom bearing them a heterocycle optionally substituted with a (C1-C6)alkyl group.

10. The method according to claim 9, wherein R13 and R14 represent, independently of one another, H or (C1-C6)alkyl.

11. The method according to claim 1, wherein R13 represents H, and R14 represents COR34, R34 representing an aryl or heteroaryl group optionally substituted with one or more groups selected from halo, (C1-C6)alkyl, OR27 and NR28R29.

12. The method according to claim 1, wherein:
X₁ represents CR² and X₂ represents CR⁵;
R² represents CONR11R12;
R11 represents H;
R12 represents a 6-membered heteroaryl group optionally substituted with one or more groups selected from halo, (C1-C6)alkyl, OR24 and NR25R26, wherein the 6-membered heteroaryl group is a pyridyl, pyridazinyl, pyrimidinyl, pyrazinyl, or triazinyl; and
R13 and R14 represent, independently of one another, H or a (C1-C6)alkyl group.

13. The method according to claim 1, wherein the compound is chosen from the group consisting of: 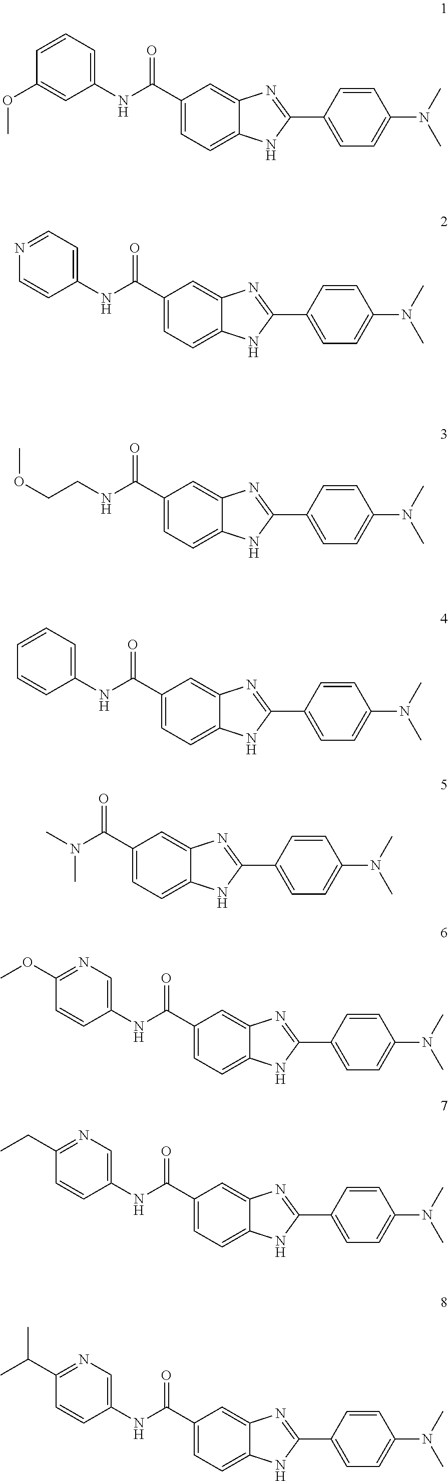 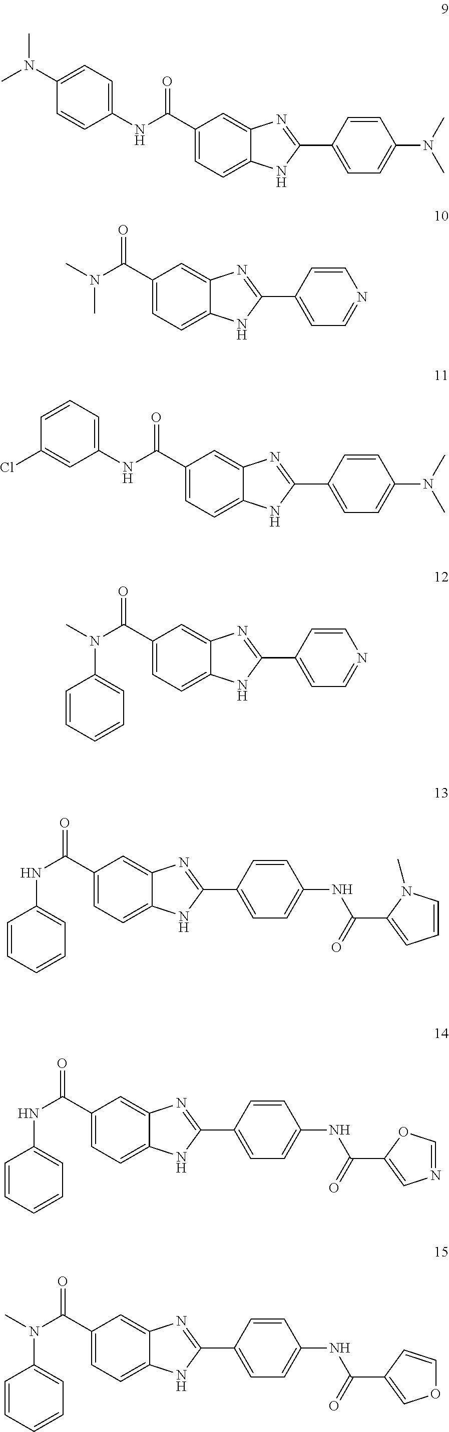 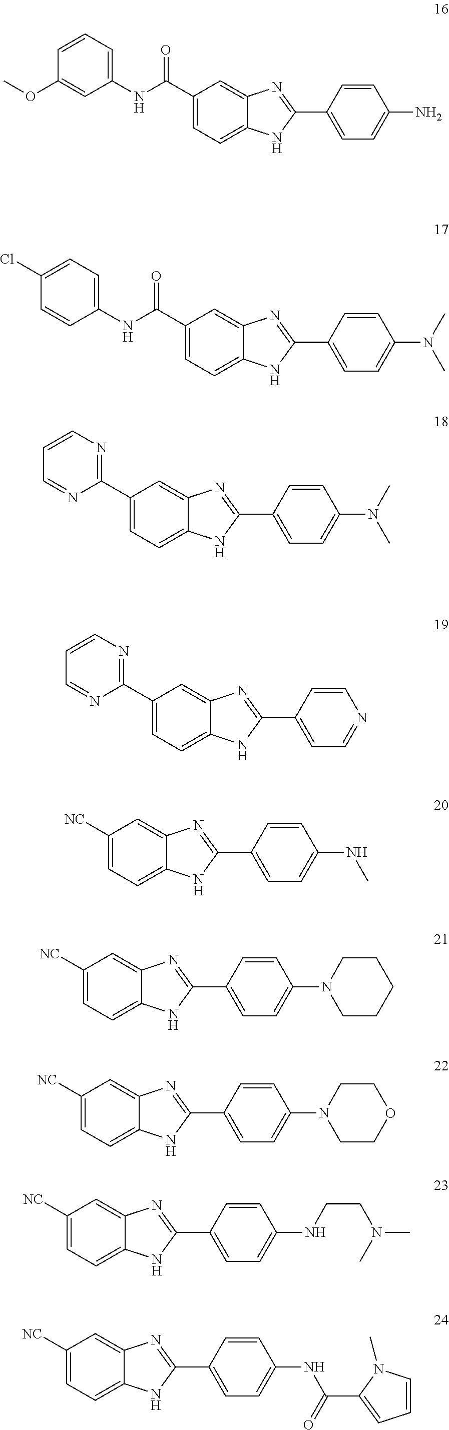 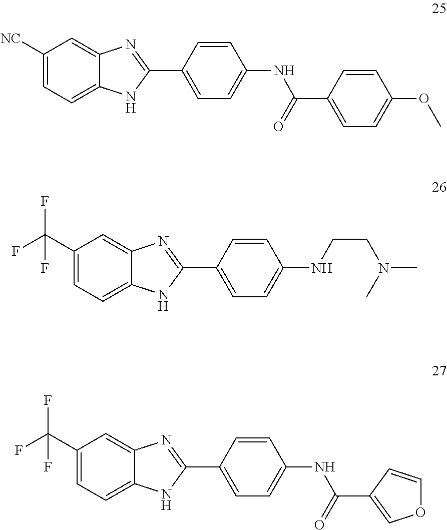 [SMILES: COc1ccc(C(=O)Nc2ccc(-c3nc4cc(C#N)ccc4[nH]3)cc2)cc1 ; CN(C)CCNc1ccc(-c2nc3cc(C(F)(F)F)ccc3[nH]2)cc1 ; O=C(Nc1ccc(-c2nc3cc(C(F)(F)F)ccc3[nH]2)cc1)c1ccoc1]
and the pharmaceutically acceptable salts thereof.

14. (canceled)

15. The method of claim 1, further comprising administering to the subject at least one other active ingredient different from the compound simultaneously, separately or sequentially,
said at least one other active ingredient being a corticosteroid; a mustard agent; a MAPK inhibitor; thalidomide; or a combined chemotherapy.

16. The method according to claim 1, wherein the histiocytosis is a histiocytosis from L, C or R group.

17. The method according to claim 1, wherein the histiocytosis is a Langerhans cell histiocytosis (LCH), a Erdheim-Chester Disease (ECD), a mixed form of Erdheim-Chester Disease and Langerhans cell histiocytosis (mixed ECD and LCH), an indeterminate cell histiocytosis (ICH), a xanthogranuloma histiocytosis (XG), or a Rosai-Dorfman disease (RDD); and wherein the craniopharyngioma is a papillary craniopharyngioma (PCP) or an adamantinomatous craniopharyngioma (ACP).

18. The method according to claim 9, wherein the heterocycle is a saturated 5- or 6-membered heterocycle.

19. The method according to claim 11, wherein the aryl is a phenyl, and wherein the heteroaryl is a 5- or 6-membered heteroaryl.

20. The method according to claim 19, wherein the 5- or 6-membered heteroaryl is furyl, thienyl, pyrrolyl, oxazolyl, isoxazolyl, thiazolyl, isothiazolyl, imidazolyl, pyrazolyl, oxadiazolyl, thiadiazolyl, triazolyl, or tetrazolyl.

21. The method according to claim 15, wherein the corticosteroid is prednisone or dexamethasone.
